(19) United States
(12) Patent Application Publication
(10) Pub. No.: US 2009/0208566 A1
(43) Pub. Date: Aug. 20, 2009

(54) Capsules Containing Seminal Material for Artificial Insemination

(76) Inventors: Daniele Vigo, Santa Giulietta (IT); Maria Luisa Torre, Pavia (IT); Massimo Faustini, Milano (IT); Eleonora Munari, Buccinasco (IT); Vincenzo Russo, Milano (IT); Ubaldo Conte, Busto Arsizio (IT)

Correspondence Address: ABELMAN, FRAYNE & SCHWAB, 666 THIRD AVENUE, 10TH FLOOR, NEW YORK, NY, 10017, US

(21) Appl. No.: 11/887,893

(22) PCT Filed: Apr. 3, 2006
(86) PCT No.: PCT/IB2006/000768

§ 371 Date: Nov. 26, 2008

(30) Foreign Application Priority Data
Apr. 4, 2005 (IT) MI2005A000552

(51) Int. Cl. A61K 9/48; A61K 9/16; B01J 13/02

(52) U.S. Cl. 424/451; 264/4.1; 424/490; 424/497

(57) ABSTRACT

Capsules or microcapsules comprising: a) a nucleus containing seminal material or the spermatozoa of animal species chosen from the group consisting of equids, buffalo, ovicaprids, canids, felids, lagomorphs, laboratory animal species chosen from mice and rats, and possibly man, b) a membrane of a bivalent or trivalent metal alginate.

### FIELD OF THE INVENTION

The present invention relates to capsules containing seminal material of animal species chosen from the class consisting of: equids, buffalo, ovicaprids, canids, felids, lagomorphs, laboratory animal species chosen from mice and rats, and possibly man, suitable for artificial insemination and the relative process for preparing said capsules or microcapsules.

### STATE OF THE ART

Instrumental insemination (II), also known as artificial insemination (Al) in zootechnics, officially tested from the second half of the 1700s, was extensively developed and applied in particular at the beginning of the 1900s. The species having had the most recourse to II is the bovine species, because of its economic importance and also precise physiological controls enable the procedure to be undertaken with considerable precision and high probability of success. Until the second half of the last century, in the bovine sector, the procedure mainly comprised the use of fresh and/or refrigerated seminal material. Instrumental insemination was undertaken at public or private facilities also using seminal material derived from external collection centres. Over the last few years, II techniques have considerably improved following the optimisation and wide diffusion of seminal material freezing techniques: cryopreservation and II have enabled excellent yields in terms of fertilization and fertility to be achieved in the bovine sector.

Over the last 20 years the development of mammalian cell encapsulation has also been a turning point in the field of reproductive technology and biotechnology in the zootechnical field. The aim of encapsulation was to entrap a population of living cells, within the boundaries of semi-permeable membranes, isolating them from the external environment: the degree of selectivity and permeability of the membrane allowed the diffusion of metabolites and nutrient substances essential for cell survival (Lim F., Sun A M. “Microencapsulated islets as bioartificial endocrine pancreas”. Science 210 (1980), 908-910).

In 1985 Nebel adopted the encapsulation technique for encapsulating bovine spermatozoa using alginates and polyamines. The method described by Nebel was advantageous in that the artificial insemination method presented some essential requisites, such as low cost, protection of seminal material from external agents, ease of handling by the operators and practicality of use. The principal aim was to improve birth rate results and to reduce phagocytosis of the spermatozoa by leucocytes, in the uterus.

This encapsulation of bovine seminal material proved to be highly complex as it comprised three stages:

1) producing a calcium alginate matrix containing the bovine spermatozoa,

2) forming a semi-permeable membrane through interfacial polymerisation with a multivalent polyamine by means of surface cross-linking of the matrix,

3) dissolving the semi-solid matrix through chelation of the calcium with sodium citrate (substitution of the calcium by sodium) and release of the seminal material. In this manner the seminal material was found to be immobilized within the calcium alginate matrix and then suspended or dispersed in sodium alginate. The polymer used influenced the persistence of the capsule in utero: poly-L-lysine capsules dissolved more rapidly than non-biodegradable polyvinylamine capsules. The capsules with a membrane consisting of protamine sulfate proved to have some advantageous characteristics: as well as adhering to the mucosas and being biodegradable, they were able to protect spermatozoa from phagocytosis and to render them less susceptible to backflow phenomena. (Nebel, R L.; Bame, J H.; Saake, R G.; Lim, F. “Microencapsulation of bovine spermatozoa”. J. Anim. Sci. 60 (1985), 1631-1639. Nebel, R L.; Saacke, R G. “Spermatozoa microencapsulation and capsule behaviour in the female tract”. Reprod. Domest. Anim. 31 (1996), 75-85. Nebel, R L; Soede, R G. “Spermatozoa microencapsulation of bovine spermatozoa”. J. Anim. Sci. 60 (1998), 1631-1639.).

A different approach to encapsulating bovine sperm cells is that described in U.S. Pat. No. RE34,326 in 1993 which uses a thermal gel characterised by being solid at ambient temperature and liquid at body temperature. The bovine seminal material can be stored for 14 days at 7° C. retaining about 50% of its mobility.

An improvement to the encapsulation technique is described in the patent EP0922451 in which a method for encapsulating pig seminal material undertaken in a single production step is given. In particular, with this encapsulation technique, a metal ion suspension is added to one of pig sperm cells followed by dropwise addition of the suspension into an aqueous sodium alginate solution. When a drop of suspension, containing the seminal material and the bivalent ions comes into contact with the sodium alginate solution, the bivalent ions diffuse towards the surface of the drop and, at the interface, cause the alginate to gel with formation of a semi-permeable membrane.

U.S. Pat. No. 6,596,310 from 2003 gives the preparation of capsules for pig spermatozoa release: The described method concerns the preliminary dilution of seminal material in a medium that prevents premature capacitation (a process indispensable for fertilization) and subsequent encapsulation of diluted seminal material. At the point of II, to achieve fertilization, it is indispensable to introduce capacitating agents into the uterus together with the capsules. This process is therefore complex and in practice is not easy to use in farming.

Although for some species, such as bovines or swine, cryopreservation or encapsulation of seminal material have allowed the II technique to be improved, the use of II in equine species is particularly problematic in that stallion seminal material is difficult to cryopreserve: once frozen, equine spermatozoa lose most of their efficiency and reproductive effectiveness. In addition, the spermatozoa of equine species are particularly sensitive to the addition of diluants; indeed, the response of the spermatozoa to dilution is an initial increase in cellular activity followed by a drastic reduction in motility and substantial structural changes to the plasma membrane. Watson in “Recent developments and concepts in the cryopreservation of spermatozoa and the assessment of their post-thawing function”. Reprod. Fertil. Dev. 7 (1995), 871-891 hypothesised that such changes are attributable to the so called “dilution effect” consisting of cell damage with loss of intracellular components and dilution of protective agents in the seminal plasma. Dilution of the semen removes proteins and antioxidants naturally present which are necessary for maintaining membrane integrity and cell function (Harrison et al., RAP.; “Bovine serum albumin, sperm motility and the dilution effect”. J. Exp. Zool. 222 (1982), 81-88; Maxwell, W M C.; Johnson, L A. “Physiology of spermatozoa at high dilution rates: the influence of seminal plasma”. Theriogenology 52 (1999), 1353-1362.)

To this is added the fact that the females of equine species have a prolonged oestrus and therefore it is difficult to establish the optimal moment for the II operation, which must be done within a few hours of the start of ovulation. Therefore, in order to guarantee fertilization in equine species, repeated II operations have so far been necessary.

For similar reasons, with other species, such as buffalo or canids, a single fertilization operation with diluted and/or refrigerated seminal material does not at the moment sufficiently guarantee optimal fertilization.

Attempts to encapsulate or microencapsulate equine seminal material or undiluted equine spermatozoa have not so far been reported in the literature. In addition to the case of equids, similar considerations can also be made for other species, such as buffalo, ovicaprids, canids, felids, lagomorphs, mice and rats and laboratory species. These species are in fact characterised by complex follicular and ovulatory dynamics and by the point of ovulation being difficult to predict.

In the specific case of the equine species, as with various others of the aforementioned species, repeated instrumental insemination operations must be resorted to, alternated by a period of time, clinically estimated to be between 12 and 48 hours with a considerable increase in farming expense.

### SUMMARY OF THE INVENTION

It has now been surprisingly found that capsules or microcapsules containing live and viable seminal material can be produced, without diluting the seminal material, in a single preparative step which preserves the genetic heritage of the male animal. Said capsules or microcapsules can be stored either at ambient temperature or under controlled refrigeration after suspending in diluents for seminal material and used for II operations.

The present invention therefore provides:

Capsules or microcapsules comprising:

a) a nucleus containing seminal material or the spermatozoa of animal species chosen from the group consisting of: equids, buffalo, ovicaprids, canids, felids, lagomorphs, laboratory animal species chosen from mice and rats, and possibly man,

b) a membrane of a bivalent or trivalent metal alginate.

A particular aspect of the present invention are capsules or microcapsules containing equine seminal material.

In this respect we have unexpectedly found and experimentally proven that encapsulation or microencapsulation of equine seminal material enables seminal material to be protected which, as is known, is characterised by a high instability. Encapsulation of equine semen, in particular in membranes of said biocompatible and biodegradable polymer, allows the prolonged and controlled release of live and viable seminal material. The gradual bioerosion of the capsules or microcapsules in the uterus (which depends on membrane thickness and characteristics) allows high concentrations of spermatozoa to be maintained within the uterus and tubes for a prolonged period of time able to cover the entire duration of oestrus, thus resulting in an increase in fertilization and fertility. Moreover the use of the encapsulated or microencapsulated seminal material of the present patent application allows a single II operation to be undertaken, to considerably reduce the number of operations in that the living and viable spermatozoa are released in a technically predetermined time period which covers the entire ovulation period.

A further aspect of the present invention is the process for preparing the aforesaid capsules or microcapsules which in particular comprise the following steps:

1) a saturated solution of a bivalent or trivalent metal ion is added to the seminal material previously taken from the donor, to obtain a suspension of seminal material;

2) the suspension from step 1) is extruded drop by drop through single or multiple needles or nozzles; the extruded drops are collected in a sodium alginate solution to obtain capsules or microcapsules of a gelatinous type which are separated by filtration and subsequently dispersed in a suitable diluent for encapsulated seminal material;

3) the capsules or microcapsules obtained in the preceding step may possibly be cross-linked on the internal and/or external surface, by relative suspension in an aqueous solution of a polyamine type cross-linking agent maintained under agitation, to give rise to rigid microcapsules or capsules.

With this method capsules of gelatinous appearance are obtained in a single step, without dilution of the seminal material, then filtered off and washed; they can be suspended in a diluting medium, with no capacitating agent, and then used for II in equine species.

The described and claimed process for forming the capsules allows the morphological, functional and genetic characteristics of the contained seminal material to remain unchanged in that the membrane protects the spermatozoa contained within from external agents and from phagocytosis.

With this type of preparation the drawbacks connected to the high sensitivity of the equine seminal material due to the effect of dilution can be avoided. In particular, from one stallion ejaculate, a high number of capsules or microcapsules containing undiluted seminal material can be produced, sufficient to obtain several doses useful for II with considerable economic advantages for the breeder.

Moreover, a single or a small number of instrumental inseminations can be successfully carried out, leading to the additional advantages of a drastic reduction in operations by the operator and the use of a reduced quantity of seminal material. The quantity of seminal material necessary for II is therefore reduced. A further advantage of the procedure is the fact that the seminal material is suitably protected and is released from the capsules or microcapsules of the present patent, within a time period programmed according to membrane thickness and based on physiological factors related to times and modes of ovulation of the animal species under consideration.

### DETAILED DESCRIPTION OF THE INVENTION

The capsules or microcapsules of the present invention preferably contain undiluted equine seminal material.

The aforesaid equine seminal material generally consists of spermatozoa at different stages of development.

The seminal material can be derived from the ejaculate or withdrawn from various parts of the male genital apparatus by means of techniques known to every person skilled in the art.

The concentration of spermatozoa in the total seminal material can be determined by direct count using a Makler camera or a Bürker camera or a cytofluorimeter or semi-automatic and automatic cell counters, and the morphological characteristics (live and dead, capacitated and not, etc.) can be determined with the evaluation techniques known to persons skilled in the art.

The nucleus (a) in addition to containing the seminal material can possibly contain a hydrophilic polymer capable of further modulating seminal fluid release at determined times.

The polymeric material, possibly added to the ejaculate forming the nucleus of the capsules of the present invention, is preferably chosen from the group consisting of: glucans, scleroglucans, mannans, galactomannans, gellans, carrageenans, pectins, polyanhydrides, polyamino acids, polyamines, xanthans, cellulose and derivatives thereof, carboxymethylcellulose, ethylcellulose, methylcellulose, hydroxypropyl cellulose, hydroxypropyl methylcellulose, polyvinyl alcohols, carboxyvinyl polymers, starches, collagens, chitins, chitosans, alginic acid, hyaluronic acid.

This hydrophilic polymer preferably constitutes between 5% and 60% by weight on the total weight of the microcapsule.

The constituent bivalent or trivalent metal alginates of the membrane (b) are preferably chosen from those of calcium, barium, strontium, zinc and trivalents from those of aluminium, iron and chromium. In accordance with a particularly preferred embodiment the capsule or microcapsule membrane of the present invention is of barium alginate.

The bivalent or trivalent metal alginate to be used for preparing the membrane (b) forms between 0.5% and 50% of the total capsule weight.

The capsules or microcapsules are generally of spheroid shape with dimensions between 0.5 and 20 mm, preferably between 1 and 10 mm. The thickness of the relative membranes is generally between 0.1 and 5 mm, preferably between 0.1 and 3 mm, even more preferably between 0.2 and 1.5 mm.

In accordance with a particularly preferred embodiment of the capsules said thickness is between 0.4 and 1 mm. The weight of the produced capsules is generally between 5 mg and 200 mg, preferably between 20 mg and 100 mg.

The capsules or microcapsules suspended in the medium can be stored either at ambient temperature or under controlled refrigeration.

Hormones and biologically active substances such as agents for stimulating maturation and preserving the activity of said equine seminal material can be carried within said capsules.

Preferably the process for preparing the capsules or microcapsules of the present invention is conducted in accordance with the following operating conditions.

The bivalent metal ion in the form of chloride or sulfate in solution is added to the seminal material suspension of step (1) until a cation concentration of between 1.0 and 1000 mmol/L, preferably between 1 and 500 mmol/L, is obtained.

In accordance with a particularly preferred embodiment a saturated barium chloride solution is added until the concentration of barium ion is preferably between 5.0 and 250 mmol/L, even more preferably between 5.0 and 100 mmol/L. Steps (1) and (2) of the process of the present invention are preferably conducted at a temperature between 5 and 40° C., even more preferably at a temperature between 20 and 30° C.

In step (2) the suspension of equine seminal material is then extruded through extruders, orifices, nozzles or needles, of dimensions between 50 μm and 50,000 μm, preferably through needles with an internal diameter preferably between 300 μm and 20,000 μm, into a stirred sodium alginate solution maintained between 10 and 200 rpm, but more preferably between 20 and 100 rpm.

The extrusion takes place by means of automatic or semi-automatic micro-encapsulators, peristaltic pumps of piston or reciprocating type, or with a syringe operated manually or by a suitable system, at a rate such as to produce between 10 and 250 drops/minute, preferably 60 drops/minute.

The ratio of volume of extruded cell suspension to alginate solution can be between 1:1 and 1:1250, preferably between 1:15 and 1:50.

The sodium alginate used in step (2) of the process of the present invention preferably presents, in a 2% aqueous solution, a viscosity between 200 cP and 20,000 cP at 25° C. The sodium alginate solution presents a concentration of between 0.01 and 5% w/v, but preferably between 0.1 and 1.0% w/v.

In step (3) of the process of the invention, said capsules can be subjected to cross-linking by means of interface polymerisation of the alginate by using cross-linking agents of polyamine type such as: protamine sulfate or phosphate, preferably in the form of a solution at concentrations between 0.01 and 5% w/v, or poly-L-lysine hydrobromide of molecular weight between 1,000 and 800,000 in aqueous solution at a concentration preferably between 0.01 and 5% w/v, or polyvinylamine at a concentration between 0.01 and 5% w/v, or chitosans of molecular weight between 15,000 and 1,000,000 at concentrations between 0.01 and 5% w/v.

The cross-linking reaction is conducted at a temperature between 5 and 40° C., preferably at 25° C. for a time period between 1 minute and 120 minutes, preferably between 3 and 30 minutes.

This procedure results in the conversion of the gelatinous membrane into a semi-permeable rigid membrane of cross-linked alginate.

After cross-linking, the capsules are recovered by filtration, then washed and suspended in a suitable maintenance medium (diluent) known to the expert of the art.

The following non-limiting examples of the procedure for preparing the capsules of the present invention are given by way of illustration.

### EXAMPLE 1

### Encapsulation of Equine Seminal Material to Obtain Immediate Release Capsules

1a) Preparation of Seminal Material

The ejaculate, collected from genetically selected stallions in accordance with techniques known to the experts of the art, is deprived of the gelatinous fraction by filtration. The classical laboratory evaluations are carried out on the ejaculate, in particular the concentration of live and viable spermatozoa is determined by means of direct count with a Makler camera.

1b) Encapsulation

A saturated barium chloride solution is added to the ejaculate, after filtering, until the concentration of the barium ion is 5 mmol/L. The resulting suspension of seminal material is extruded through needles (26 G×½″, 0.45×13 mm) into a sodium alginate solution of medium viscosity (3500 cP) at 0.5% w/v in a culture medium consisting of an aqueous solution containing 60 g/l of glucose and 1.2 g/l of sodium bicarbonate; the solution has a pH of 7.4. The ratio of seminal material to sodium alginate solution is 1:25. The extrusion takes place drop by drop by means of a syringe, at a temperature of 25° C. The barium ions react with the sodium alginate to form in 30 minutes a membrane of barium alginate at the interface of the single extrusion drops. Capsules are obtained which are collected by filtration, washed twice in a diluant for equine semen and suspended in an aliquot thereof.

Spheroid shaped capsules are obtained whose characteristics are given hereinafter:

Average weight: 59.2 mg

Total diameter: 6.1 mm

Diameter of nucleus: 5.0 mm

Membrane thickness: 0.5 mm

The encapsulated seminal material is stored at a temperature of 17° C. for 72 hours.

The viability of the encapsulated spermatozoa, determined every 24 hours by the vital stain Trypan Blue, is given hereinafter (on at least 200 cells per specimen):

Time 0: 69.5%

Time 24 hours: 63.2%

Time 48 hours: 19.8%

Time 72 hours: 16.4%

The viability of the encapsulated spermatozoa is significantly higher than that of the diluted spermatozoa (see reference example 3)

### EXAMPLE 2

### Encapsulation of Equine Seminal Material to Obtain Prolonged Release Capsules

2a) Preparation of Equine Seminal Material

The ejaculate was collected in accordance with techniques known to the experts of the art, as given in example 1a).

The classical laboratory evaluations known to the experts of the art are carried out on the ejaculate and the concentration of live and viable spermatozoa is determined by means of direct count with a Makler camera.

2b) Encapsulation

A saturated barium chloride solution is added to the ejaculate until the barium ion concentration is 15 mmol/L. The resulting suspension is extruded through needles (26 G×½″, 0.45×13 mm) into a sodium alginate solution of medium viscosity (3500 cP) at 0.5% w/v in a culture medium consisting of an aqueous solution containing 60 g/l of glucose and 1.2 g/l of sodium bicarbonate (Sigma Aldrich), pH 7.4. The ratio of seminal material to sodium alginate solution is 1:25. The extrusion takes place drop by drop by means of a syringe, at a temperature of 25° C. The barium ions react with the sodium alginate to form in 30 minutes a membrane of barium alginate at the interface of the single extrusion drops. Capsules are obtained which are collected by filtration, washed twice in culture medium and suspended in an aliquot thereof.

Spheroid shaped capsules are obtained whose characteristics are given hereinafter:

Average weight: 92.9 mg

Total diameter: 6.5 mm

Diameter of nucleus: 5.0 mm

Membrane thickness: 0.7 mm

The encapsulated seminal material is stored at a temperature of 17° C. for 72 hours.

The viability of the encapsulated spermatozoa, determined every 24 hours by the vital stain Trypan Blue, is given hereinafter (on at least 200 cells per specimen):

Time 0: 65.9%

Time 24 hours: 64.3%

Time 48 hours: 17.1%

Time 72 hours: 11.6%

The viability of the encapsulated spermatozoa is significantly higher than that of the diluted spermatozoa (see reference example 3).

### REFERENCE EXAMPLE 3

### Dilution of Equine Seminal Material According to the Known Art

3a) Preparation of Equine Seminal Material

The ejaculate was collected in accordance with techniques known to the experts of the art, as given in example 1a).

The classical laboratory evaluations known to the experts of the art are carried out on the ejaculate and the concentration of live and viable spermatozoa is determined by means of direct count with a Makler camera.

3b) Dilution

The ejaculate is diluted in a 1:10 ratio with a commercially available diluent for equine semen in accordance with techniques known to the expert of the art.

The viability of the spermatozoa stored at 17° C., determined by the vital stain Trypan Blue, is given hereinafter (on at least 200 cells per specimen):

Time 0: 59.0%

Time 24 hours: 49.7%

Time 48 hours: 10.7%

Time 72 hours: 5.7%

Simple dilution of the spermatozoa brings about a significant reduction of vitality compared to spermatozoa encapsulated in both types of capsule.

1. A capsule or microcapsule comprising:
a) a nucleus containing seminal material or the spermatozoa of animal species chosen from the group consisting of: equids, buffalo, ovicaprids, canids, felids, lagomorphs, laboratory animal species chosen from mice and rats, and possibly man,
b) a membrane of a bivalent or trivalent metal alginate.

2. The capsule or microcapsules as claimed in claim 1 containing undiluted equine seminal material in the nucleus (a).

3. The capsule or microcapsules as claimed in claim 2 containing equine seminal material consisting of spermatozoa at different stages of development.

4. The capsules or microcapsules as claimed in claim 1, wherein the nucleus (a) contains a hydrophilic polymer.

5. The capsules or microcapsules as claimed in claim 4, wherein said hydrophilic polymer is chosen from glucans, scieroglucans, mannans, galactomannans, gellans, carrageenans, pectins, polyanhydrides, polyamino acids, polyamines, xanthans, cellulose and derivatives thereof, carboxymethylcellulose, ethylcellulose, methylcellulose, hydroxypropyl cellulose, hydroxypropyl methylcellulose, polyvinyl alcohols, carboxyvinyl polymers, starches, collagens, chitins, chitosans, alginic acid, hyaluronic acid.

6. The capsule or microcapsule as claimed in claim 4 wherein said polymer constitutes between 5% and 60% by weight on the total weight of the microcapsule.

7. The capsule or microcapsules as claimed in claim 1, wherein the constituent bivalent or trivalent metal alginates of the membrane (b) are preferably chosen from those of calcium, barium, strontium, zinc and trivalents from those of aluminium, iron and chromium.

8. The capsule or microcapsule as claimed in claim 7, wherein the membrane of the capsules or microcapsules of the present invention is of barium alginate.

9. The capsules or microcapsules as claimed in claims 1, wherein the bivalent or trivalent metal alginate constitutes from 0.5% and 50% by weight of the total capsule or microcapsule weight.

10. The capsules or microcapsules as claimed in claims 1, presenting dimensions between 0.5 and 20 mm.

11. The capsules or microcapsules as claimed in claim 10, presenting dimensions between 1 and 10 mm.

12. The capsules or microcapsules as claimed in claims 1, having a thickness between 0.1 and 5 mm.

13. The capsules or microcapsules as claimed in claim 12, wherein said thickness is between 0.1 and 3 mm.

14. The capsules or microcapsules as claimed in claims 1, wherein said thickness is between 0.2 and 1.5 mm.

15. The capsules or microcapsules as claimed in claim 14, presenting dimensions between 0.4 and 1 mm.

16. The capsules or microcapsules as claimed in

17. The capsules or microcapsules as claimed in claim 16, weighing between 20 and 100 mg.

18. Process for preparing the capsules or microcapsules claimed in claims 1, comprising the following steps:
1) a saturated solution of a bivalent or trivalent metal ion is added to the seminal material previously taken from the donor, to obtain a suspension of seminal material;
2) the suspension from step 1) is extruded drop by drop through single or multiple needles or nozzles; the extruded drops are collected in a sodium alginate solution to obtain capsules or microcapsules of gelatinous type which are separated by filtration and then dispersed in a suitable diluent for encapsulated seminal material;
3) the capsules or microcapsules obtained in the preceding step may be crosslinked on their internal and/or external surface, by relative suspension in an aqueous solution of a polyamine type cross-linking agent maintained under agitation to give rise to rigid microcapsules or capsules.

19. Process as claimed in claim 18, wherein the bivalent metal ion in the form of chloride or sulfate in solution is added to the seminal material suspension of step (1) until a cation concentration of between 1.0 and 1000 mmol/L is obtained.

20. Process as claimed in claim 19, wherein said concentration is between 1 and 500 mmol/L.

21. Process as claimed in claim 19, wherein said salt is barium chloride at concentrations between 5.0 and 250 mmol/L.

22. Process as claimed in claim 21,

22. Process as claimed in claim 21, wherein said barium chloride concentration is between 5.0 and 100 mmol/L.

23. Process as claimed in 18, wherein steps (1) and (2) of the process are conducted at temperatures between 5 and 40° C.

24. Process as claimed in claim 23, wherein said temperature is between 20 and 30° C.

25. Process as claimed in claim 18, wherein in step (2) the suspension of equine seminal material is then extruded through extruders, orifices, nozzles or needles, of dimensions between 50 μm and 50,000 μm.

26. Process as claimed in claim 25, wherein said extruders, orifices, nozzles or needles present an internal diameter preferably between 300 μm and 20,000 μm.

27. Process as claimed in claim 18, wherein said extrusion in step (2) is achieved by means of automatic or semi-automatic micro-encapsulators, peristaltic pumps of piston or reciprocating type, or with a syringe operated manually at a rate such as to produce between 10 and 250 drops/minute, while maintaining the sodium alginate solution stirring at a speed between 10 and 200 rpm.

28. Process as claimed in claim 27, wherein said rate is 60 drops/minute and the stirring speed is between 20 and 100 rpm.

29. Process as claimed in claim 18, wherein the ratio of extruded cell suspension to alginate solution volume is between 1:1 and 1:250.

30. Process as claimed in claim 29, wherein said ratio is between 1:15 and 1:50.

31. Process as claimed in claim 18, wherein the sodium alginate used in step (2) presents, in a 2% aqueous solution, a viscosity of between 200 cP and 20,000 cP at 25° C.

32. Process as claimed in claim 18, wherein the alginate solution presents a concentration of between 0.01 and 5.0% w/v.

33. Process as claimed in claim 32 wherein said concentration is between 0.1 and 1.0% w/v.

34. Process as claimed in claim 18, wherein said cross-linking agent of polyamine type is selected from the group consisting of protamine sulfate, poly-L-lysine hydrobromide, polyvinylamine and chitosans.

35. Process as claimed in claim 34, wherein said cross-linking agent is protamine sulfate or phosphate in an aqueous solutions at concentrations between 0.01 and 5% w/v.

36. Process as claimed in claim 34, wherein said cross-linking agent is poly-L-lysine hydrobromide of molecular weight between 1000 and 800,000 in the form of an aqueous solution at concentrations between 0.01 and 5% w/v.

37. Process as claimed in claim 34, wherein said cross-linking agent is polyvinylamine at a concentration between 0.01 and 5% w/v.

38. Process as claimed in claim 34, wherein said cross-linking agent is a chitosan with a molecular weight between 15,000 and 1,000,000 at concentrations between 0.01 and 5% w/v.

39. Process as claimed in claim 18, wherein the cross-linking reaction of step (3) is conducted at a temperature between 5 and 40° C. for a time period between 1 minute and 120 minutes.

40. Process as claimed in claim 39, wherein said cross-linking is conducted at 25° C. for a time period between 3 and 30 minutes.

41. Process as claimed in claim 18, wherein the capsules derived from step (2) or (3) are recovered by filtration, then washed and suspended in a diluent and stored either at ambient temperature or under controlled refrigeration.
